(19) United States
(12) Patent Application Publication
(10) Pub. No.: US 2008/0145417 A1
(43) Pub. Date: Jun. 19, 2008

(54) PHARMACEUTICAL FORMULATION FOR THE EFFICIENT ADMINISTRATION APOMORPHINE, 6AR-(-)-N-PROPYL-NORAPOMORPHINE AND THEIR DERIVATIVES AND PRO-DRUGS THEREOF

(75) Inventors: Hakan Wikstrom, Groningen (NL); Durk Dijkstra, Bedum (NL); Thomas Ivo Franciscus Hubert Cremers, Groningen (NL)

Correspondence Address: OBLON, SPIVAK, MCCLELLAND MAIER & NEUSTADT, P.C., 1940 DUKE STREET, ALEXANDRIA, VA, 22314, US

(73) Assignee: AXON BIOCHEMICALS B.V., Groningen (NL)

(21) Appl. No.: 12/033,646

(22) Filed: Feb. 19, 2008

Related U.S. Application Data
(62) Division of application No. 10/478,692, filed on Apr. 30, 2004.

(30) Foreign Application Priority Data
Jun. 8, 2001 (SE) 0102036-1

(51) Int. Cl. A61K 9/50; A61K 31/4745; A61P 15/00; A61K 9/16; A61K 9/20

(52) U.S. Cl. 424/451; 514/290; 424/490; 424/474

(57) ABSTRACT

An efficient pharmaceutical formulation for the treatment of an affliction selected from the group consisting of Parkinson's disease, restless legs syndrome and erectile dysfunction. Said composition comprises at least one member selected from the group consisting of apomorphine, 6aR-(−)-N-propyl-norapomorphine and their derivatives and pro-drugs thereof in the form of the base or the pharmaceutically acceptable salts or solvates thereof as an active ingredient in a pharmaceutical preparation suited for oral or intraduodenal administration.

### TECHNICAL FIELD

This invention relates to the efficient administration of a formulation of apomorphine, 6aR-(−)-N-propyl-norapomorphine and their derivatives and pro-drugs thereof for treating i.a. Parkinson's disease (PD), restless legs syndrome (RLS), psychogenic male erectile dysfunction (MED), and female sexual dysfunction, or the like afflictions.

### BACKGROUND OF THE INVENTION

Apomorphine has been used to treat Parkinsonian patients. See, for example, Hagell P. and Odin P., J. Neurosci Nurs Feb, 33(1):21-34, 37-8 (2001); Deffond et al., J. Neurology, Neurosurgery, and Psychiatry 56:101-103 (1993) and Durif et al., Clinical Neuropharmacology 16(2):157-166 (1993). Additionally, apomorphine has been considered for the treatment of alcoholism, schizophrenia, dystonia musculorum deformans, hallucinations, migraine headaches, hiccups, Huntington's chorea, tardative dyskinesia, and more recently male erectile dysfunction.

Parkinson's disease is a progressive, neurodegenerative disorder caused by a loss of the cell bodies of dopaminergic (DA-ergic) neurons from the substantia nigra and degeneration of nerve terminals in the striatum resulting in low levels of DA in the substantia nigra and corpus striatum. Parkinson's disease is characterized by chronic, progressive motor dysfunction and its main symptoms are tremor at rest, muscle rigidity and a decrease in the frequency of voluntary movements (hypokinesia) with difficulty in stopping, starting and turning when walking. A persistent tremor is superimposed on hypertonicity of opposing muscle groups and initiation of movements becomes increasingly difficult and slow. In advanced stages, patients' movements become virtually “frozen”, and patients are unable to care for themselves. Studies have shown that the symptoms of Parkinson's disease appear when the striatal DA content is reduced to 20-40% of normal.

As Parkinson's disease is associated with a loss of DA from the striatum, it is commonly treated with drugs which replace DA, the most commonly used of these being levodopa. Levodopa is converted by dopa decarboxylase into DA in the brain and it is this DA which exerts a therapeutic effect. Levodopa has to be administered in large and frequent doses. In addition, the production of DA in peripheral tissues gives rise to unwanted side-effects.

Accordingly, levodopa is normally given in combination with other drugs to enhance the effects of levodopa in the brain and minimize its peripheral effects. In particular, levodopa is usually given in combination with a peripheral dopadecarboxylase inhibitor, which cannot cross the blood-brain barrier, such as carbidopa, which inhibits the breakdown of levodopa to DA outside the brain, thereby reducing peripheral unwanted effects. The inhibitor also ensures that a relatively large amount of an oral dose of levodopa reaches the brain and thus enables the dose of levodopa to be reduced which also reduces peripheral side-effects. In addition, a peripheral DA antagonist, which does not penetrate the blood-brain barrier, such as domperidone, may also be administered to reduce the nausea and vomiting side-effects of levodopa.

In addition to the side-effects mentioned above, further undesirable effects are associated with the prolonged use of levodopa. In particular, many patients develop involuntary choreiform movements, which are the result of excessive activation of DA receptors. These movements usually affect the face and limbs and can become very severe. Such movements disappear if the dose of levodopa is reduced but this causes rigidity to return. Moreover, the margin between the beneficial and the unwanted effect appears to become progressively narrower as the period of levodopa treatment increases. The traditional method of combating this effect is to increase the frequency of administration of levodopa whilst keeping the overall dose steady. This approach reduces end-of-dose deterioration and diminishes the likelihood of the patient developing the dyskinesias that occur with high peak doses.

A further complication of long-term levodopa treatment is the development of rapid fluctuations in clinical state where the patient switches suddenly between mobility and immobility for periods ranging from a few minutes to a few hours. This phenomenon is known as the “on-off effect”, the “on” state being the preferred state during which nearly normal motor functioning can be attained and the “off” state being characterized by dystonic postures during periods of decreased mobility. Indeed, this effect can produce such an abrupt loss of mobility that the patient may suddenly stop while walking or be unable to rise from a chair in which he had sat down normally a few moments earlier. This effect is commonly unaffected by manipulation of the dose of levodopa and may require treatment with alternative drugs. In addition to the above long-term side-effects of levodopa treatment, it has been found that the effectiveness of levodopa gradually declines with time until it is no longer effective. Also, an increased incidence of malignant melanoma has been observed in patients undergoing treatment with levodopa and it has therefore been suggested that treatment with levodopa may be linked with the development of malignant melanoma. Accordingly, the use of levodopa in the treatment of Parkinson's disease is far from ideal.

An alternative approach to the treatment of Parkinson's disease is the use of drugs that mimic the action of DA. Such drugs are collectively known as DA agonists because they directly stimulate DA receptors within the DA-deficient nigrostriatal pathway. Unlike levodopa, DA agonists do not need to be converted in the brain to active compounds. Also, DA agonists are effective in patients in the advanced stages of Parkinson's disease when levodopa is no longer effective because they act directly on the DA receptors and are therefore unaffected by the lack of DA-producing nerve cells in such patients. However, the action of such DA agonists on the DA receptors also causes unwanted DA-ergic effects, such as nausea, vomiting and extrapyramidal effects, which can be debilitating and some DA agonists, such as apomorphine, are associated with further undesirable side-effects, especially when high doses are used, such as sedation, respiratory depression, hypotension, bradycardia, sweating and yawning. The severity and nature of such side-effects can be affected by the mode of administration of the drug. For instance, studies involving apomorphine have investigated a variety of routes for administration of this drug. However, oral administration of apomorphine tablets has required high doses to achieve the necessary therapeutic effect. Also, long-term studies involving such oral forms were stopped after 7-10 days due to unexplained rises in blood urea nitrogen. Sub-lingual administration of apomorphine tablets caused severe stomatitis on prolonged use with buccal mucosal ulceration in half the patients treated. Intranasal administration produced transient nasal blockage, burning sensation and swollen nose and lips and, in some of the patients tested, had to be withdrawn because of what was considered to be chemical inflammation of the nasal mucosa (Zaleska, B. et al., Neurol. Neurochir. Pol. 33:1297-1303, 1999).

Accordingly, so far, the only satisfactory way of administering apomorphine for treating Parkinson's disease, which avoids high first pass metabolism, has been found to be sub-cutaneous administration and, thus, the only commercially available formulation of apomorphine is a liquid for subcutaneous injection or subcutaneous infusion. Even so, subcutaneous administration does not avoid the normal DA agonist side-effects, such as nausea and vomiting and subcutaneous administration, whether by injection or infusion, is not easy to accomplish, particularly by patients whose motor functions are already impaired, and therefore requires training of patients and caretakers. Also, the injection site must be changed every 12 hours to minimize risks of skin discoloration and nodules forming. In view of these problems, it is not surprising that the use of DA agonists, such as apomorphine, in the treatment of Parkinson's disease has been largely confined to the treatment of “off” periods caused by levodopa therapy despite the obvious clinical benefits of such drugs over levodopa.

It is apparent from the above that it would be highly desirable from a clinical point of view to find a way of administering DA agonists, such as apomorphine, 6aR-(−)-N-propylnorapomorphine and their derivatives and pro-drugs thereof, which is efficient and easy for the patient to use.

Restless Legs Syndrome (RLS; see also Glasauer F E, Spinal Cord 2001 March; 39(3):125-33) is a well-defined symptom complex and is frequently associated with sleep disturbance and a recognized family history. It occurs either as idiopathic RLS or in association with many medical, neurological or vascular disorders. The neurological examination and routine investigations in idiopathic RLS are normal. Polysomnography supports the diagnosis of RLS by documenting the associated sleep disturbances and periodic limb movements in sleep (PLMS). There is supportive evidence that RLS is a Central Nervous System (CNS) dysfunction, suggesting widespread involvement of the descending dopaminergic pathways, possibly originating in the diencephalon or upper brainstem. This is corroborated by the successful treatment of RLS with DA agents, sedatives, and neurotransmitters. However, RLS can also occur with spinal disorders and spinal cord lesions implying the existence of a spinal generator. The incidence of RLS in pregnancy is well known and its association with vascular disorders supports another mechanism in some patients. The primary treatment of RLS is largely symptomatic and quite effective with DA agents, DA agonists, opioids and other drugs affecting various neurotransmitters. The treatment of RLS associated with various diseases is aimed at the correction of the underlying pathological or deficiency states. Antidepressant medications frequently precipitate or worsen the condition of RLS. It has been reported that nocturnal subcutaneous apomorphine infusion has a beneficial effect on sleep quality in booth Parkinson's disease and restless legs syndrome (RLS; see Reuter I, Ellis C M, Ray Chaudhuri K, Acta Neurol Scand 1999 September; 100(3):163-7). The study by Reuter et al. suggests that overnight apomorphine infusion may be effective in overcoming refractory nocturnal disabilities in selected patients with Parkinson's disease and restless legs syndrome.

Impotence or male erectile dysfunction (ED) is defined as the inability to achieve and sustain an erection sufficient for intercourse. Impotence in any given case can result from psychological disturbances (psychogenic), from physiological abnormalities in general (organic), from neurological disturbances (neurogenic), hormonal deficiencies (endocrine) or from a combination of the foregoing. These descriptions are not exact, however. There is currently no standardized method of diagnosis or treatment. As used herein, psychogenic impotence is defined as functional impotence with no apparent overwhelming organic basis. It may be characterized by an ability to have an erection in response to some stimuli (e.g., masturbation, spontaneous nocturnal, spontaneous early morning, video erotica, etc.) but not others (e.g., partner or spousal attention). The specific mechanisms by which apomorphine acts to produce an erectile response in a human patient are not yet completely understood, however. Sublingual apomorphine (Uprima®) is presently marketed in some European countries for treating male erectile dysfunction.

Apomorphine has been shown to have very poor oral bioavailability. (See, for example, Baldessarini et al., in Gessa et al., eds., Apomorphine and Other Dopaminomimetics, Basic Pharmacology, Vol. 1, Raven Press, N.Y. (1981), pp. 219-228). Thus, the search is continuing for an effective oral apomorphine treatment of PD, RLS and psychogenic impotence in male patients as well as for diagnostic methods that can identify such patients.

### SUMMARY OF THE INVENTION

By this invention a pharmaceutical formulation for the administration of apomorphine, 6aR-(−)-N-propyl-norapomorphine and their derivatives and pro-drugs thereof is provided by means of which the low oral bioavailability of apomorphine, 6aR-(−)-N-propyl-norapomorphine (NPA) and their derivatives and pro-drugs thereof can be avoided.

The invention is based on the surprising finding in an animal experiment that intraduodenally administered apomorphine is pharmacologically very potent in comparison with apomorphine administered in the conventional oral way ending in the stomach. The same is true for NPA. On basis thereof the present invention provides a pharmaceutical formulation containing apomorphine, 6aR-(−)-N-propyl-norapomorphine and their derivatives and pro-drugs thereof in the form of the base or a pharmaceutically acceptable salt or solvate thereof as an active ingredient in a pharmaceutical formulation for oral/intraduodenal administration either directly or by passing the gastric compartment (the stomach=gastrum) intact by being provided with an enteric coating and being quickly dissolved and absorbed in the duodenum/small intestine, or in a formulation with controlled release of the active ingredient (e.g. by being encapsulated in a plastic skeleton, which may be biodegradable).

In several reports, the use of intraduodenal administration of aqueous solutions of drugs have shown several advantageous features as compared to oral administration (into gastrum) of both tablets, suspensions and solutions (e.g. Watari et al., J. Pharmacokinet. Biopharm, October 1983 11 (5), p. 529-545). Especially, the variation of drug plasma concentration was substantially reduced by using the intraduodenal route, mainly due to avoidance of the effect of variations in gastric emptying times. Furthermore, the compound apomorphine is extremely sensitive to oxidation and will decompose in solutions which are in contact with atmospheric air. Through the present invention, the drawbacks mentioned above are eliminated to a large extent.

### DETAILED DESCRIPTION OF THE INVENTION

As indicated above, the present invention provides a pharmaceutical formulation for the treatment of Parkinson's disease, restless legs syndrome, male erectile dysfunction and female sexual dysfunction, which composition comprises at least one member selected from the group consisting of apomorphine, 6aR-(−)-N-propyl-norapomorphine and their derivatives and pro-drugs thereof in the form of the base, a pharmaceutically acceptable salt or solvate of either of these as the active ingredient in a pharmaceutical formulation suited for oral/intraduodenal administration.

According to a preferred embodiment the pharmaceutical formulation according to the invention is in the form of a compressed tablet or granules for oral administration comprising said active ingredient together with appropriate excipients and adjuvants and being provided with an enteric coating dissolving in the small intestine (duodenum, jejunum and/or ileum), e.g. duodenum.

Apomorphine is a dopamine D1 and D2 receptor agonist that has a recognized use as an anti-parkinsonian drug when administered subcutaneously in about a 5 mg dose. For the purposes of the present invention, apomorphine is administered orally in an amount sufficient to treat PD, RLS and/or ED in humans. The dose needed to treat these different conditions may differ with the condition and with the individual patient.

This is attributable to the preferred absorption of apomorphine, 6aR-(−)-N-propyl-norapomorphine and their derivatives and pro-drugs thereof in a limited segment of the human gastrointestinal tract, i.e., the small intestine (e.g. the duoenum).

The instant invention provides a dosage form for apomorphine, 6aR-(−)-N-propyl-norapomorphine and their derivatives and pro-drugs thereof which utilizes an enteric coated, rapidly disintegrating/dissolving tablet consisting of apomorphine, 6aR-(−)-N-propyl-norapomorphine and their derivatives and pro-drugs thereof. Such a dosage form provides a convenient method of once or more a day patient dosing in conjunction with conventional dosage forms of apomorphine, 6aR-(−)-N-propyl-norapomorphine and their derivatives and pro-drugs thereof.

The formulations of the present invention may contain other additional agents which are well-known to those skilled in the art in connection with pharmaceutical compositions containing apomorphine. As examples of such agents may be mentioned anti-emetics (e.g. domperidone), pro-kinetic agents (e.g. domperidone), stabilizers, anti-oxidants, preserving agents and pH-regulating agents.

Excipients and adjuvants to be used in the pharmaceutical formulations according to the invention in the form of a compressed tablet or granules may include (1) fillers to add bulk and improve compressibility, e.g., lactose, starch, sugar-alcohols, cellulose derivatives, calcium sulfate or phosphate, (2) disintegrants to disintegrate the dosage form, e.g., starch, sodium starch glycolate, cellulose derivatives, alginates, gums, effervescent mixtures, (3) binders to form granules or improve compressibility, e.g., gums, sugars, starch, cellulose derivatives, alginates, polyvinylpyrrolidone, (4) lubricants to reduce friction, e.g., stearic acid, metallic stearates, high melting point waxes, talc, (5) agents to improve dissolution, e.g., surfactants, alkaline buffers and (6) glidants to improve flow, e.g., starch, talc, silicate.

When preparing the tablets/granules a tablet/granule core is first prepared by compressing a mixture of the active ingredient(s), excipients, adjuvants and possible other additives. The enteric coating layer is then applied on said tablet/granule core by conventional coating techniques such as, for instance, pan-coating or fluidized bed coating using solutions or film-forming polymers in water and/or suitable organic solvents or by using suspensions of such polymers. Examples of such film-forming polymers are shellac, cellulose acetate phthalate, hydroxypropyl methyl cellulose, polyvinyl acetate phthalate, carboxymethyl ethyl cellulose and co-polymers synthesized from methacrylic acid and methacrylic acid methyl ester such as the product sold under the trade name Eudragit®S by Rohm Pharma, Darmstadt, Germany.

Solvents to be used in this connection include, for instance, methanol, ethanol, isopropanol and methylene chloride.

The solutions or suspensions of the film-forming agent may optionally contain pharmaceutically acceptable plasticizers such as, for instance, polyethylene glycol, castor oil, glycerol, propylene glycol, and phthalic acid esters.

Dispersants, such as talc, may also be included in the enteric coating layer.

According to a variant of this embodiment the compressed tablet/granule provided with an enteric coating dissolving in duodenum/small intestine exhibits a further, outer layer comprising a said active ingredient along with appropriate excipients and adjuvants to give an immediate release dose in combination with the delayed dose.

In accordance with another embodiment of the present invention the pharmaceutical formulation comprises a mixture of said active ingredient and appropriate excipients and adjuvants enclosed in a capsule dissolving in duodenum/small intestine. Preferably said mixture is in the form of a solution of the active ingredient in a solvent such as water or a pharmaceutically acceptable organic solvent or oil together with e.g. an anti-emetic agent, a stabilizer, an anti-oxidant, a preserving agent and/or a pH-regulating agent. The capsule itself should be of a material which is resistant to gastric juice but rapidly dissolves when approaching and entering duodenum.

In accordance with a further embodiment of the present invention the pharmaceutical preparation is in the form of enteric coated granules enclosed in a capsule dissolving in the stomach (gastrum), releasing the enteric coated granules, which have an optimal size to flow with the gastric contents into duodenum and disintegrate there or further downstream the small intestine, under controlled release of the active ingredient.

The active ingredient, when used in a pharmaceutical formulation in which it is not present in solution, should be in micronized form, e.g. having a particle size within the range of from 0.1 to 20 μm, preferably from 0.1 to 5 μm. Such enteric coated particles can preferably be enclosed in a capsule, which rapidly disintegrates in the gastric juice. The freed particles, which withstand the gastric juice due to their enteric coating, have an optimal size to flow into the duodenum together with the gastric content on gastric emptying. In duodenum, these particles disintegrate at a controlled rate, which is dependent on the formulation chosen for coating of such particles.

According to a further embodiment of the present invention the pharmaceutical formulation is in a form suited for administration intraduodenally by an intraduodenal catheter through the abdominal wall of a patient or by a naso-duodenal catheter.

In this embodiment the active ingredient or ingredients is preferably dissolved in a carrier such as water or a pharmaceutically acceptable organic solvent or oil. However, suspensions of the active ingredient(s) in a carrier are also contemplated.

In view of the fact that apomorphine and its derivatives are sensitive to oxidation, the formulations of the present invention should be prepared and stored under exclusion of oxygen including avoidance of contact with atmospheric air.

The pharmaceutical formulations according to the invention contain, as the active ingredient or ingredients, at least one member of the following groups of substances:

A) Apomorphine, 6aR-(−)-N-propyl-norapomorphine (NPA), symmetric di-(C₂-C₅)alkanoyl esters of aporphines and NPA and the pharmaceutically acceptable salts thereof, and the dibenzoyl ester of apomorphine and NPA and the pharmaceutically acceptable salts thereof.

B) Aporphine pro-drugs disclosed by International Patent Application No. PCT/SE01/ (claiming priority from Swedish Patent Application No. 0002934-8, filed on Aug. 17, 2000) and having the general formula:

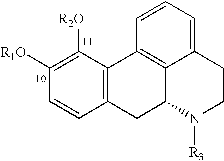

wherein
one of R₁ and R₂ is hydrogen or acetyl and the other one is selected from the group consisting of (C₃-C₂₀)alkanoyl; halo(C₃-C₂₀)alkanoyl; (C₃-C₂₀)alkenoyl; (C₄-C₇)cycloalkanoyl; (C₃-C₆)cycloalkyl (C₂-C₁₆)alkanoyl; aroyl which is unsubstituted or substituted by 1 to 3 substituents selected from the group consisting of halogen, cyano, trifluoromethanesulphonyloxy, (C₁-C₃)alkyl and (C₁-C₃)alkoxy, which latter may in turn be substituted by 1 to 3 halogen atoms; aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, which latter may in turn be substituted by 1 to 3 halogen atoms; and heteroarylalkanoyl having one to three heteroatoms selected from O, S and N in the heteroaryl moiety and 2 to 10 carbon atoms in the alkanoyl moiety and which is unsubstituted or substituted in the heteroaryl moiety by 1 to 3 substituents selected from the group consisting of halogen, cyano, trifluoromethanesulphonyloxy, (C₁-C₃)alkyl, and (C₁-C₃)alkoxy, which latter may in turn be substituted by 1 to 3 halogen atoms; and R₃ is methyl; and the physiologically acceptable salts thereof. Symmetric di-(C₂-C₅)alkanoyl esters and the di-benzoyl ester of aporphines have been described and reports of bioavailability of such esters have been presented, but the overall result was disappointing. As an example, the di-pivaloyl ester pro-drug was much less active than the parent compound apomorphine itself.

The alkanoyl groups of the symmetric di-(C₂-C₈)alkanoyl esters of apomorphine may be of a straight or branched chain. Such symmetric di-alkanoyl esters include, e.g. the diacetyl, di-propionyl, di-butyryl and di-pivaloyl esters of apomorphine.

One preferred group of aporphine pro-drugs to be used in the present invention and being disclosed by PCT/SE01/ comprises mono-(C₂-C₅)alkanoyl esters of apomorphine in which the alkanoyl group may be of a straight or branched chain. Examples of such esters include mono-acetyl, mono-butyryl and mono-pivaloyl apomorphine.

Another preferred group of aporphine pro-drugs to be used in the present invention and being disclosed by PCT/SE01/ comprises asymmetrical di-alkanoyl esters of apomorphine, wherein one of the alkanoyl groups is acetyl and the other is (C₃-C₅) alkanoyl, the chain of which may be straight or branched. Examples of such esters include propionyl, acetyl apomorphine, butyryl, acetyl apomorphine, isobutyryl, acetyl apomorphine, isopropanoyl, acetyl apomorphine and pivaloyl, acetyl apomorphine.

According to a further aspect of the present invention there is provided a method of treating an affliction selected from the group consisting of Parkinson's disease, restless legs syndrome, male erectile dysfunction and female sexual dysfunction, which method comprises administering orally/intraduodenally to a patient in need of treatment a pharmaceutical formulation according to the present invention as identified above in an effective ameliorating amount.

The invention will now be further described by means of a number of examples which are not to be construed as limiting the scope of the present invention.

### EXAMPLE 1

### Preparation of Tablets Containing Apomorphine Hydrochloride

Core tablets are prepared by mixing apomorphine hydrochloride with microcrystalline cellulose, sodium starch glycolate, corn starch, talc and magnesium stearate in suitable proportions according to acceptable pharmaceutical manufacturing practices. The finished blend is screened and convex core tablets/granules are compressed by direct compression using a suitable tablet press yielding tablets/granules.

Compressed core tablets/granules thus prepared are enteric coated by means of a suspension formed from Eudragit® S, 12.5% suspension in isopropanol; polyethylene glycol 6000, 33% aqueous solution; talc and isopropanol/acetone 1:1. The core tablets/granules are enteric coated by spraying the above Eudragit-S suspension onto their surfaces as tablets/granules rotate in a conventional coating pan to produce an even, uninterrupted surface distribution of the coating.

### EXAMPLE 2

### Preparation of Tablets Containing Apomorphine Derivatives

Microcrystalline cellulose (MCC) (PH 112, Eur. Ph; from OPG Groothandel B.V., Utrecht, The Netherlands) was mixed with apomorphine hydrochloride (APO), monopivaloyl-apomorphine (MPA) (prepared according to WO 02/14279A1) (UVPA) (from Sigma) respectively. In the mixtures the ratio MCC/apomorphine was 5/1 w/w (i.e. 83% MCC/17% apomorphine derivative). The mixtures were homogenised by vortexing and shaking.

Compaction of the mixtures into circular biconvex tablets (12 tablets) with a diameter of 4 mm and a weight of 25-30 mg was performed using an ESH hydraulic press (Hydro Mooi, Appingedam, The Netherlands). A compaction pressure of ca 100 MPa was used for all the tablets.

After compaction tablets were provided with layers of enteric coating. This coating consisted of Eudragit® L30 (from Röhm, Darmstadt, Germany), which is a 30% w/v suspension of methacrylic acid/methylmethacrylate copolymer. This substance is insoluble at acidic pH hut readily soluble at neutral and basic pH. 5 g of this suspension was mixed with water (4 g), talc (0.75 g), Citroflex® (triethyl citrate from Fluka, Buchs, Switzerland) (0.15 g), and silicon antifoam solution (from Boom, Meppel, The Netherlands) (0.05 g). This was stirred for ca one hour before use. The coating procedure was then as follows; The tablets were placed in a flat circular sieve with a diameter of 45 mm. The tablets were preheated to a temperature of ca 40-45° C. using a hair dryer. Then a drop (30-50 μl) of the coating liquid was added to the sieve and the tablets were stirred with a glass rod under a stream of hot air until the water had evaporated. This was repeated 8 times, yielding tablets with a uniform layer of enteric coating. The tablets were then left over night to dry.

TABLE 1
Mixtures used for the tablets and the average weight of the
tablets before and after coating

| Apomorphine | | | |
|---|---|---|---|
| derivative | | Weight of tablet | |
| (mg) | MCC (mg) | Before coating (mg) | After coating (mg) |
| APO (67.9) | 335 | 29.6 | 37.4 |
| NPA (66.5) | 336 | 29.9 | 38.1 |
| MPA (65.2) | 336 | 29.5 | 39.3 |

### EXAMPLE 3

### Preparation of Tablets Containing Apomorphine Hydrochloride (APO) (12%) in Biodegradable PLG Polymer and mono-pivaloyl-N-propyl-noraporphine (MPNPA)

96 mg Microcrystalline cellulose (MCC) (PH 112, Eur. Ph.) (from OPG Groothandel B.V., Utrecht, The Netherlands) was mixed with 4.2 mg MPPA. The mixture was homogenised by vortexing and shaking.

Compaction of the mixture into three circular biconvex tablets with a diameter of 4 mm and a weight of 25-30 mg was performed using an ESH hydraulic press (Hydro Mooi, Appingedam, The Netherlands). From APO in PLG polymer tablets with an approximate weight of 40 mg were made in a similar way. A compaction pressure of ca 100 MPa was used for all the tablets. The weight of the tablets was determined on an analytical balance (Mettler-Toledo).

After compaction tablets were provided with layers of enteric coating. This coating consisted of Eudragit® L30 (from Röhm, Darmstadt, Germany), which is a 30% w/v suspension of methacrylic acid/methylmethacrylate copolymer. This substance is insoluble at acidic pH but readily soluble at neutral and basic pH. 5 g of this suspension was mixed with water (4 g), talc (0.75 g), Citroflex® (triethyl citrate from Fluka, Buchs, Switzerland) (0.15 g) and silicon antifoam solution (from Boom, Meppel, The Netherlands) (0.05 g). This was stirred for ca one hour before use. The coating procedure was then as follows; The tablets were placed in a flat circular sieve with a diameter of 45 mm. The tablets were preheated to a temperature of ca 40-45° C. using a hair dryer. Then a drop (30-50 μl) of the coating liquid was added to the sieve and the tablets were stirred with a glass rod under a stream of hot air until the water had evaporated. This was repeated 8 times, yielding tablets with a uniform layer of enteric coating. The tablets were then left over night to dry. The weight of the tablets was determined on an analytical balance (Mettler-Toledo)

TABLE 2
Weights of tablets before and after coating, respectively

| | Weight before coating | Weight after coating |
|---|---|---|
| Tablet type | (mg) | (mg) |
| MPNPA | 28.32 ± 1.16 | 34.39, 35.92 |
| APO + PLG | 36.97 ± 0.88 | 44.06 ± 1.23 |

### Pharmacological Experiments

1. Behavioural Experiment—Injection into Duodenum

Apomorphine hydrochloride (4 mg/kg or 5 mg/kg) and its mono pivaloyl ester (4.6 mg/kg or 4.9 mg/kg) and N-propylnoraporphine (NPA; 5 mg/kg) were injected with a bolus injection into the duodenum of rats. These rats had been operated 1-14 days before the experiment. A plastic tubing was introduced entering through the duodenum wall at about the mid section and bent in such a way that it had its duct directed downwards (i.e. aiming downstream towards the jejunum and being about 2 cm long). An experienced scientist observed the animals for the entire period of pharmacological activity, scoring the behavior and emphasizing the following details: yawning, sniffing, chewing, licking, rearing, grooming, penile grooming, locomotor activity and stereotypy. The total duration of action where one or several of these behaviors were present was scored.

| | dose | |
|---|---|---|
| Compound | (mg/kg) | duration of dopaminergic behavior (min) |
| Apomorphine | 4.0 | intense stereotypy (5-75); less intense |
| Hydrochloride | | stereotypy (75-85); dur (90) |
| Apomorphine | 5.0 | intense stereotypy (5-40); less intense |
| Hydrochloride | | stereotypy (40-90); dur (95) |
| Apomorphine | 5.0 | intense stereotypy (5-45); less intense |
| Hydrochloride | | stereotypy (45-55); dur (60) |
| Mono-Piv-Apo | 4.6 | intense stereotypy (5-105); less intense |
| | | stereotypy (105-115); dur (120) |
| Mono-Piv-Apo | 5.9 | intense stereotypy (5-105); less intense |
| | | stereotypy (105-130); dur (135) |
| Mono-Piv-Apo | 5.9 | intense stereotypy (5-80); less intense |
| | | stereotypy (80-90); dur (120) |
| NPA | 5.0 | intense stereotypy (5 min-9 h); dur (>9 h) |

As a control experiment, apomorphine hydrochloride (4 mg/kg) was administered orally to the same rat. Very weak dopaminergic stimulation was observed and the time period in which these effects were observed was 10-20 min.

### 2. Behavioural Experiment—Entero-Coated Pill

One entero-coated tablet prepared as described in Example 1 and containing about 5 mg of NPA hydrochloride was placed under anesthesia (isoflurane) in the throat of a rat and pushed down the throat with a blunt instrument. Within five minutes, the rat was awake and exploring the cage. After about 3 to 4 hours, the rat began to show dopaminergic stimulatory signs like sniffing, chewing, penile licking, grooming and stereotypy with rearing, locomotor activity, intense sniffing and also licking. This stereotypy lasted for more than 24 hours.

3. Microdialysis Experiment (Striatum) with One Entero-Coated Tablet Containing NPA Hydrochloride

One entero-coated tablet prepared as described in Example 1 and containing about 5 mg of NPA hydrochloride was administered to a rat in the way described in Pharmacological Experiment 2 and a standard microdialysis was carried out.

After an initial decrease in dopamine release, after about two hours dopamine release is diminished, However, after about four hours, which is about the time needed for passage through the stomach and uncoating in the small intestine, dopamine release is maximally decreased to about 20 percent of control values, This effect is lasting for several hours until the experiment was stopped, At this time, the rat performed stereotypy behavior, which by experience is equal to a maximum decrease in dopamine release.

4. Microdialysis Experiment (Striatum) with One Entero-Coated Tablet Containing Mono-Pivaloyl-Apomorphine Base

Pharmacological Experiment 2 was repeated but using an entero-coated tablet containing about 5 mg of mono-pivaloyl-apomorphine base instead of NPA hydrochloride.

After about 60 minutes, dopamine release decreases down to of a maximal decrease of 20% (i.e. 80% of control values), Dopamine release is back to control values after about eight hours.

5. Microdialysis Experiment (Striatum) with One Entero-Coated Tablet Containing about 1 mg of mono-pivaloyl-N-propyloraporphine (MPNPA) Base

Pharmacological Experiment 2 was repeated but using an enterocoated tablet prepared as described in Example 3 containing about 1 mg of mono-pivaloyl-N-propyl-noraporphine (MPNPA) base instead of NPA hydrochloride.

Dopamine release decreases continuously between one hour and four hours (maximal decrease down to 30% of controls) and then slowly increasing to a value of 80% of controls at 18 hours after application of the pill. An intense stereotypy was noted between four hours and eight hours from injection.

### 6. Behavioural Experiment

A behavioural experiment was carried out using three tablets (each containing about 5 mg of apomorphine hydrochloride embedded in a buyer degradable PLG plastic matrix and prepared as described in Example 3) applied under anesthesia in the throat of a rat and pushed down the throat with a blunt object.

Weak signs of behavioural stimulation like chewing, sniffing, grooming, penile licking and some motor activity were noted during the time of the experiment (10 hours). It is thus clear that small amounts of apomorphine have be released from the tablets and absorbed in the small intestine. The experiment was ended by anesthetizing the rat with isoflurane and sampling blood directly from the heart of the rat. The brain was also taken out and homogenized in 60 percent CH₃CN/water and the solids were removed by centrifugation. In order to investigate if the tablets were still to be found, the intestinal system from the stomach to the descending colon was checked in detail. Two tablets were found in the colon and one tablet was found in the descending colon embedded in a preformed piece of stools. These three tablets were dried overnight in vacuum desiccator and weighed (34.6 mg, 35.5 mg and 35.6 mg). Before administration the mean weight of these tablets was about 37 mg, which means that the weight after passing the intestinal system is about the same as the weight before coating.

Thus, a more efficient formulation would be to use an enterocoated capsule filled with apomorphine, an apomorphine derivative or a biodegradable formulation like that used for the tablets in the behavioural experiment above.

What we claim is:

1. A method of treating male erectile dysfunction in a subject in need thereof comprising administering an effective amount of a pharmaceutical formulation comprising at least one active ingredient selected from the group consisting of apomorphine, 6aR-(−)-N-propyl-norapomorphine and their derivatives and pro-drugs thereof in the form of the base or the pharmaceutically acceptable salts or solvates thereof.

2. The method of claim 1, wherein said administering is oral or intraduodenal.

3. The method of claim 1, wherein said pharmaceutical formulation is in a form of a compressed tablet or granule and further comprises one or more excipients and/or adjuvants and wherein said tablet or granule has an enteric coating.

4. The method of claim 3, wherein said tablet or granule further comprises an outer layer outer layer comprising said active ingredient along with appropriate excipients and adjuvants.

5. The method of claim 3, wherein said pharmaceutical formulation is in a form of a compressed tablet.

6. The method of claim 1, wherein aid pharmaceutical formulation is in a form of a capsule and comprises a mixture of said active ingredient and one or more excipients and/or adjuvants enclosed therein.

7. The method of claim 6, wherein said mixture in the form of granules.

8. The method of claim 1, wherein said pharmaceutical formulation is in a form of an enteric coated granule enclosed in a capsule dissolving in the stomach (gastrum), releasing the enteric coated granules, which have an optimal size to flow with the gastric contents into duodenum and disintegrate there or further downstream the small intestine, under controlled release of the active ingredient.

9. The method of claim 1, wherein said active ingredient has a particle size ranging from 0.1 to 20 μm.

10. The method of claim 1, wherein said active ingredient has a particle size ranging from 0.1 to 5 μm.

11. The method of claim 1, wherein said administering is intraduodenal and is administered by an intraduodenal catheter through the abdominal wall of a patient or by a nasoduodenal catheter.

12. The method of claim 1, wherein the active ingredient is a pharmaceutically acceptable salt of apomorphine or 6aR-(−)-N-propyl-norapomorphine (NPA).

13. The method of claim 1, wherein the active ingredient is a aporphine pro-drug and is selected from the group consisting of symmetric di-(C₂-C₅)alkanoyl esters of apomorphine and NPA and pharmaceutically acceptable salts thereof and the di-benzoyl ester of apomorphine and NPA and the pharmaceutically acceptable salts thereof.

14. The method of claim 1, wherein the active ingredient is a aporphine pro-drug and is selected from the group consisting of compounds having the formula: 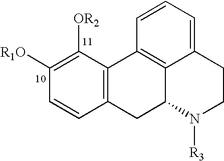
wherein
one of R₁ and R₂ is hydrogen or acetyl and the other one is selected from the group consisting of (C₃-C₂₀)alkanoyl; halo-(C₃-C₂₀)alkanoyl; (C₃-C₂₀)alkenoyl; (C₄-C₇)cycloalkanoyl; (C₃-C₆)-cycloalkyl(C₂-C₁₆)alkanoyl; aroyl which is unsubstituted or substituted by 1 to 3 substituents selected from the group consisting of halogen, cyano, trifluoromethanesulphonyloxy, (C₁-C₃)alkyl and (C₁-C₃)alkoxy, in which the latter may in turn be substituted by 1 to 3 halogen atoms; aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, in which the latter may in turn be substituted by 1 to 3 halogen atoms; and hetero-arylalkanoyl having one to three heteroatoms selected from O, S and N in the heteroaryl moiety and 2 to 10 carbon atoms in the alkanoyl moiety and which is unsubstituted or substituted in the heteroaryl moiety by 1 to 3 substituents selected from the group consisting of halogen, cyano, trifluoromethane-sulphonyloxy, (C₁-C₃)alkyl, and (C₁-C₃)alkoxy, in which the latter may in turn be substituted by 1 to 3 halogen atoms; and
R₃ is methyl; and the physiologically acceptable salts thereof.

15. The method of claim 14, wherein the aporphine pro-drug is selected from the group consisting of mono-(C₂-C₅)alkanoyl esters of apomorphine and pharmaceutically acceptable salts thereof.

16. The method of claim 14, wherein the aporphine pro-drug is selected from the group consisting of asymmetrical di-alkanoyl esters of apomorphine, wherein one of the alkanoyl groups is acetyl and the other is a (C₃-C₆)-alkanoyl group, and pharmaceutically acceptable salts thereof.

17. A method of treating female sexual dysfunction in a subject in need thereof comprising administering an effective amount of a pharmaceutical formulation comprising at least one active ingredient selected from the group consisting of apomorphine, 6aR-(−)-N-propyl-norapomorphine and their derivatives and pro-drugs thereof in the form of the base or the pharmaceutically acceptable salts or solvates thereof.

18. The method of claim 17, wherein said administering is oral or intraduodenal.

19. The method of claim 17, wherein said pharmaceutical formulation is in a form of a compressed tablet or granule and further comprises one or more excipients and/or adjuvants and wherein said tablet or granule has an enteric coating.

20. The method of claim 19, wherein said tablet or granule further comprises an outer layer outer layer comprising said active ingredient along with appropriate excipients and adjuvants.

21. The method of claim 19, wherein said pharmaceutical formulation is in a form of a compressed tablet.

22. The method of claim 17, wherein aid pharmaceutical formulation is in a form of a capsule and comprises a mixture of said active ingredient and one or more excipients and/or adjuvants enclosed therein.

23. The method of claim 22, wherein said mixture in the form of granules.

24. The method of claim 17, wherein said pharmaceutical formulation is in a form of an enteric coated granule enclosed in a capsule dissolving in the stomach (gastrum), releasing the enteric coated granules, which have an optimal size to flow with the gastric contents into duodenum and disintegrate there or further downstream the small intestine, under controlled release of the active ingredient.

25. The method of claim 17, wherein said active ingredient has a particle size ranging from 0.1 to 20 μm.

26. The method of claim 17, wherein said active ingredient has a particle size ranging from 0.1 to 5 μm.

27. The method of claim 17, wherein said administering is intraduodenal and is administered by an intraduodenal catheter through the abdominal wall of a patient or by a nasoduodenal catheter.

28. The method of claim 17, wherein the active ingredient is a pharmaceutically acceptable salt of apomorphine or 6aR-(−)-N-propyl-norapomorphine (NPA).

29. The method of claim 17, wherein the active ingredient is a aporphine pro-drug and is selected from the group consisting of symmetric di-(C₂-C₅)alkanoyl esters of apomorphine and NPA and pharmaceutically acceptable salts thereof and the di-benzoyl ester of apomorphine and NPA and the pharmaceutically acceptable salts thereof.

30. The method of claim 17, wherein the active ingredient is a aporphine pro-drug and is selected from the group consisting of compounds having the formula: 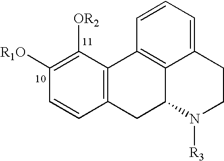
wherein
one of R₁ and R₂ is hydrogen or acetyl and the other one is selected from the group consisting of (C₃-C₂₀)alkanoyl; halo-(C₃-C₂₀)alkanoyl; (C₃-C₂₀)alkenoyl; (C₄-C₇)cycloalkanoyl; (C₃-C₆)-cycloalkyl(C₂-C₁₆)alkanoyl; aroyl which is unsubstituted or substituted by 1 to 3 substituents selected from the group consisting of halogen, cyano, trifluoromethanesulphonyloxy, (C₁-C₃)alkyl and (C₁-C₃)alkoxy, in which the latter may in turn be substituted by 1 to 3 halogen atoms; aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, in which the latter may in turn be substituted by 1 to 3 halogen atoms; and hetero-arylalkanoyl having one to three heteroatoms selected from 0, S and N in the heteroaryl moiety and 2 to 10 carbon atoms in the alkanoyl moiety and which is unsubstituted or substituted in the heteroaryl moiety by 1 to 3 substituents selected from the group consisting of halogen, cyano, trifluoromethane-sulphonyloxy, (C₁-C₃)alkyl, and (C₁-C₃)alkoxy, in which the latter may in turn be substituted by 1 to 3 halogen atoms; and
R₃ is methyl; and the physiologically acceptable salts thereof.

31. The method of claim 30, wherein the aporphine pro-drug is selected from the group consisting of mono-(C₂-C₅)alkanoyl esters of apomorphine and pharmaceutically acceptable salts thereof.

32. The method of claim 30, wherein the aporphine pro-drug is selected from the group consisting of asymmetrical di-alkanoyl esters of apomorphine, wherein one of the alkanoyl groups is acetyl and the other is a (C₃-C₆)-alkanoyl group, and pharmaceutically acceptable salts thereof.
